(19) United States
(12) Patent Application Publication
(10) Pub. No.: US 2006/0217665 A1
(43) Pub. Date: Sep. 28, 2006

(54) SURGICAL INSTRUMENT SEAL ASSEMBLY AND TRIPLE LEAD THREAD

(75) Inventor: Michael Prosek, Jacksonville, FL (US)

Correspondence Address: Mark James Young, 12086 Fort Caroline Rd., Unit 202, Jacksonville, FL, 32225, US

(73) Assignee: LAPAROSCOPIC PARTNERS LLC, Jacksonville, FL (US)

(21) Appl. No.: 11/164,324

(22) Filed: Nov. 18, 2005

Related U.S. Application Data
(60) Provisional application No. 60/629,014, filed on Nov. 18, 2004.

(51) Int. Cl. A61M 5/178

(52) U.S. Cl. 604/167.02

(57) ABSTRACT

A surgical instrument has an hourglass instrument seal operably coupled to the interior of the valve seal assembly. The hourglass instrument seal includes a top flange, a free floating lower flange and a rippled junction adjoining a top conical portion and bottom conical portion. An anti-inversion assembly biases the top flange apart from the lower flange. A tilt subassembly enables pivotal movement of the seal assembly using a ball and socket. Additionally, the cap housing may include a ball socket for slidably engaging the lower spherical section of the tilt assembly. A duckbill valve includes a pair of flaps, each having a plurality of reinforcing ribs. A fluid port is disposed at an acute upward angle relative to the channel. A cannula tube includes a plurality (e.g., three) independent parallel sets of evenly spaced threads.

### RELATED APPLICATION

This application claims the benefit of priority to U.S. Provisional Application No. 60/629,014 filed Nov. 18, 2004, the entire contents of which are incorporated herein and made a part hereof.

### FIELD OF THE INVENTION

This invention relates generally to a surgical instrument, and, more specifically, to a cannula having an hourglass shaped seal, a pivoting ball socket assembly, an insufflation gas port angled to facilitate accessibility and prevent occlusion, and a triple-lead thread to securely engage tissue while minimizing or preventing leakage of insufflation fluid from a surgical site when an instrument with a diameter within a determined range is inserted into, manipulated and withdrawn from the cannula vertically straight or at an angle relative to the central axis of the cannula.

### BACKGROUND

An important feature of a cannula is an arrangement of seals to prevent leakage of insufflation fluid through the cannula when instruments of varying sizes are inserted into, manipulated within or withdrawn from the cannula. In a variety of surgical procedures, a cannula is positioned with its distal end inside the patient and its proximal end outside the patient. One or more medical instruments are inserted through the cannula into the patient. For example, each of a sequence of instruments (including an endoscope) can be inserted through the cannula into the patient and then withdrawn (in the opposite direction) out from the patient and cannula. While inserted, an instrument may be manipulated at various angles to perform the procedure. During surgery, the body cavity, such as the abdomen, is insufflated with a fluid, typically carbon dioxide gas, under pressure to provide space between internal organs and bodily tissue.

During such procedures, seals in the cannula prevent fluid from escaping from within the patient. One seal (referred to herein as a “fluid seal”) prevents fluid escape from the cannula when no instrument occupies the cannula's channel. A fluid seal is typically comprised of a flapper valve, duckbill valve, trumpet valve or other valve, which is biased in a closed position at times when no instrument occupies the cannula's channel to provide a fluid seal preventing fluid flow through the channel at when an instrument is not inserted in the cannula. When the distal end of an instrument is inserted into the channel of the cannula and the instrument is advanced through the channel toward the patient, the instrument forces open the fluid seal (e.g., by displacing the flexible slits of a duckbill valve or displacing the trap door of a flapper valve). While the instrument is inserted and the fluid seal is open, another seal (referred to herein as an “instrument seal”) prevents fluid leakage. When the distal end of the instrument is removed from the channel of the cannula, the fluid seal returns to a closed position, providing a fluid seal.

As discussed above, another seal (i.e., the “instrument seal”) provides a fluid seal around an inserted instrument's outer periphery to prevent fluid flow through the channel of the cannula when the instrument is inserted. Conventional instrument seals consist of a washer-shaped ring of flexible material, such as an elastomer, with a central aperture sized to accommodate the cylindrical shaft of a surgical instrument. Because instruments of varying diameters (e.g., 5 mm, 7 mm, 10 mm, and 12 mm) are often inserted into the same cannula during a single surgical procedure, maintenance of a fluid-tight seal often requires use of a sizing solution such as a converter (or adapter) to downsize the opening, or an elastic (i.e., stretchable) seal with an opening capable of accommodating each instrument diameter used in the procedure.

Unfortunately, however, conventional sizing solutions have shortcomings. Use of converters is time consuming, inconvenient and costly. Conventional elastic seals stretch awkwardly when a large diameter instrument is inserted, increasing the risk that the seal will rupture, tear or otherwise fail. Additionally, an elastic seal stretched to engage a large diameter instrument tends to tightly grip the instrument, resist forward motion, invert when the instrument is withdrawn, and interfere with smooth fluid motion of the instrument. Furthermore, tilting, pivoting and otherwise angularly maneuvering an inserted instrument tends to obliquely stretch the seal opening, thereby risking leakage and structural failure.

Another problem with a conventional cannula is the position and orientation of the gas insufflation port. Typically the port extends perpendicular from the cannula channel. An engaged conduit for supplying fluid extends outwardly from the port. To avoid an occlusion, such as by kinking, the conduit sags and is bent gradually. Often, this arrangement interferes with manipulation and use of the instrument.

Yet another problem with a conventional cannula is that the threads do not securely engage tissue. Insecure threading is conducive to leakage, trauma, and compromising delicate and precision procedures.

Although attempts have been made to provide a cannula which facilitates insufflation, securely engages tissue and maintains the integrity of a fluid-tight seal for a range of instrument sizes, in various angular positions, known cannulas provided to date have failed to address the full range of surgeons' needs. The invention is directed to overcoming one or more of the problems as set forth above.

### SUMMARY OF THE INVENTION

To overcome one or more of the problems as set forth above, in one aspect of the invention, a surgical instrument comprised of a valve seal assembly is disclosed. The valve seal assembly has an interior and an exterior. An hourglass instrument seal (i.e., an instrument seal having an hourglass shape) is operably coupled to the interior of the valve seal assembly. The hourglass instrument seal may include an upper flange operably coupled to the interior of the valve seal assembly, a free floating lower flange, a top conical portion, a bottom conical portion and a rippled junction adjoining the top conical portion and bottom conical portion.

A surgical instrument according to principles of the invention may also include a tilt subassembly and a cap housing, with a cap top having a concave lower surface disposed at the proximal end of the valve seal assembly, and a tilt cap with a convex upper surface adapted to slidably engage the concave lower surface of the cap top. The tilt assembly may further include a lower spherical section. Additionally, the cap housing may include a ball socket for slidably engaging the lower spherical section of the tilt assembly. Such an arrangement facilitates pivotal movement of the seal assembly.

A surgical instrument according to principles of the invention may further include a fluid seal comprised of a duckbill valve. The duckbill valve includes a pair of flaps, each having a plurality of reinforcing ribs.

In another aspect of the invention, a surgical instrument according to principles of the invention has a valve seal assembly with a central channel, a proximal end and a distal end. The surgical instrument may include a fluid port operably coupled to the valve seal assembly in fluid communication with the channel. The port may have a free end and be disposed at an acute angle relative to the channel, with the free end of the port being angled toward the proximal end of the valve seal assembly.

A surgical instrument according to principles of the invention may further include a cannula tube operably coupled to the valve seal assembly. The cannula tube may include a threaded section having a plurality of independent parallel sets of threads. In one embodiment, the plurality of independent parallel sets of threads includes triple lead threads. The plurality of independent parallel sets of threads may start equidistant apart.

### BRIEF DESCRIPTION OF THE DRAWINGS

The foregoing and other aspects, objects, features and advantages of the invention will become better understood with reference to the following description, appended claims, and accompanying drawings, where:

FIG. 1 provides an exterior side view of an exemplary assembled surgical instrument according to principles of the invention;

FIG. 2 provides an exterior side view of an exemplary assembled surgical instrument according to principles of the invention;

FIG. 3 provides a top exterior view of an exemplary assembled cannula according to principles of the invention.

FIG. 4 provides an exploded exterior view of an exemplary cannula according to principles of the invention;

FIG. 5 provides an exploded sectional view of an exemplary cannula according to principles of the invention;

FIG. 6 provides a sectional view of an assembled exemplary cannula according to principles of the invention;

FIG. 7 provides a sectional view of an exemplary tilting sub-assembly with a cap top of a cap housing in a final position on the tilting sub-assembly according to principles of the invention;

FIG. 8 provides an exploded side view of an exemplary tilting sub-assembly according to principles of the invention;

FIG. 9 provides an exploded perspective view of an exemplary tilting sub-assembly according to principles of the invention;

FIG. 10 provides a section view of an exemplary seal cap assembly and tilting sub-assembly in a neutral or centered position according to principles of the invention;

FIG. 11 provides a section view of an exemplary seal cap assembly and tilting sub-assembly in a tilted position according to principles of the invention;

FIG. 12 provides a section view of an exemplary surgical instrument with an exemplary seal cap assembly and tilting sub-assembly in a tilted position and a small diameter surgical instrument inserted in place;

FIG. 13 provides a section view of an exemplary tilting sub-assembly with a large diameter surgical instrument inserted according to principles of the invention;

FIG. 14 provides a top perspective view of an exemplary instrument seal according to principles of the invention;

FIG. 15 provides a bottom perspective view of an exemplary instrument seal according to principles of the invention;

FIG. 16 provides a first side view of an exemplary instrument seal according to principles of the invention;

FIG. 17 provides a second view (rotated 30 degrees clockwise from the first side view of FIG. 16) of an exemplary instrument seal according to principles of the invention;

FIG. 18 provides a side sectional view of an exemplary instrument seal according to principles of the invention;

FIG. 19 provides a bottom perspective view of an exemplary fluid seal in the form of a duckbill valve according to principles of the invention; and

FIG. 20 provides a top perspective view of an exemplary fluid seal in the form of a duckbill valve according to principles of the invention.

Those skilled in the art will appreciate that the figures are not intended to be drawn to any particular scale. The invention is not limited to the exemplary embodiments depicted in the figures or the shapes, relative sizes, proportions or materials shown in the figures.

### DETAILED DESCRIPTION

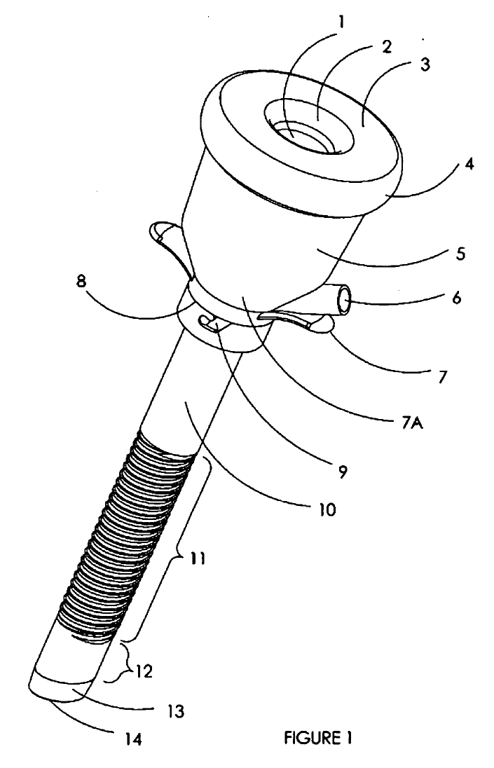

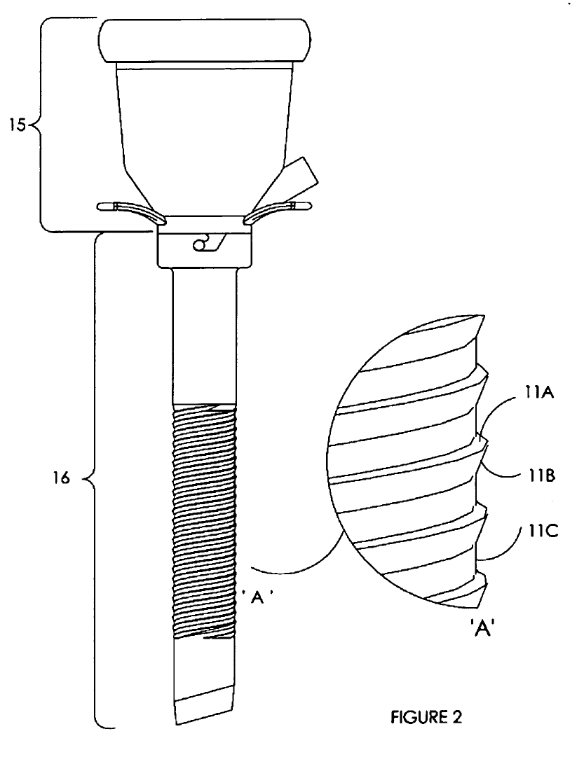

With reference to the drawings, wherein like numerals represent like features, an exterior of an exemplary assembled surgical instrument according to principles of the invention is shown in FIGS. 1 and 2. In general, the exemplary surgical instrument includes a valve seal assembly upper body portion 15 (referred to herein interchangeably as the “valve seal assembly” and “upper body portion”) releasably coupled to a lower body portion cannula tube 16 (referred to herein interchangeably as the “cannula tube” and “lower body portion”).

Referring to FIG. 1, details of the exterior of an exemplary assembled cannula according to principles of the invention are shown. An instrument through bore or channel 1 is provided from the center of the upper surface of the cap top 3 (i.e., the proximal end) extending the entire instrument length. The instrument entrance radius 2 at the proximal end provides a gradually tapered opening to facilitate viewing an instrument seal within the device and inserting an instrument. A cap radius flange 4 provides a smooth gripping surface to facilitate manually attaching and detaching the cannula tube 16 from or to the surgical instrument valve seal assembly upper body portion 15. Attachment is achieved with a twist-lock detail 9. An abutment 8 defines the interface between the upper body 15 and a cannula tube 16 portions. A pair of grip wings 7 are provided for finger tip control. A finger/thumb grip area 7a is also provided at about 90 degrees from finger grip wings 7 for an alternative or supplemental grip control.

An insufflation gas port 6 is provided in fluid communication with the channel 1. A stopcock (not shown) may be affixed, such as by bonding or with an industry standard Luer Lock attachment. The port 6 enables introduction of insufflation fluid between the distal end of the instrument and an internal fluid seal, which is described more fully below. As the gas port 6 is angled upwardly towards the proximal end, the port 6 provides ample room for maneuvering the instrument without kinking and occluding an attached conduit.

Advantageously, the cannula tube 16 includes a smooth upper cylindrical portion 10 and a mid portion 11 with triple lead anchoring threads 11A-C, as shown in FIG. 2. The triple lead threads 11A-C are comprised of three, independent, parallel sets of threads that start approximately at equal intervals (e.g., 120 degrees) apart and spiral around the mid portion 11. The triple lead threads 11A-C provide more secure anchoring than single-lead threads of conventional cannulas. Quadruple lead threads, other triple-lead threads and greater multi-thread arrangements also come within the scope of the invention.

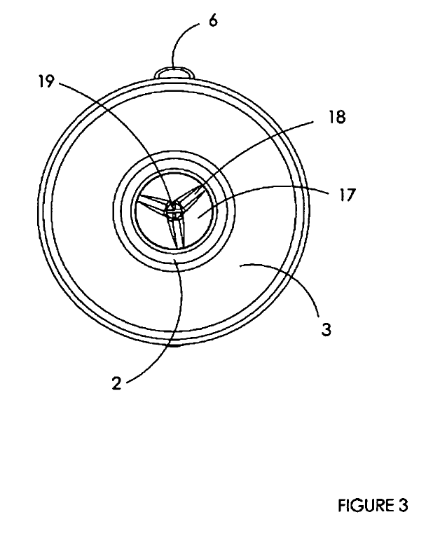

A top exterior view of an exemplary assembled cannula according to principles of the invention is shown in FIG. 3. As discussed above, an instrument throughbore or channel 1 is provided from the center of the upper surface of the cap top 3 (i.e., the proximal end) extending the entire instrument length. The instrument entrance radius 2 at the proximal end provides a gradually tapered opening to facilitate viewing an instrument seal within the device and inserting an instrument. A conical surface 17, an expanding trough 18 and an aperture 19 in an hourglass-shaped instrument seal 31, are also shown. Additionally, the gas port 6 is shown.

Towards the distal end, the cannula tube 16 has a smooth diameter cylindrical portion providing a tissue dilation area 12. A tissue dilation bevel 13 smoothly transitions between the distal end and the dilation area 12. Furthermore, a tip dilation angle 14 provides a leading edge at the distal end of the cannula tube 16 to facilitate introduction through an incision of a tissue layer.

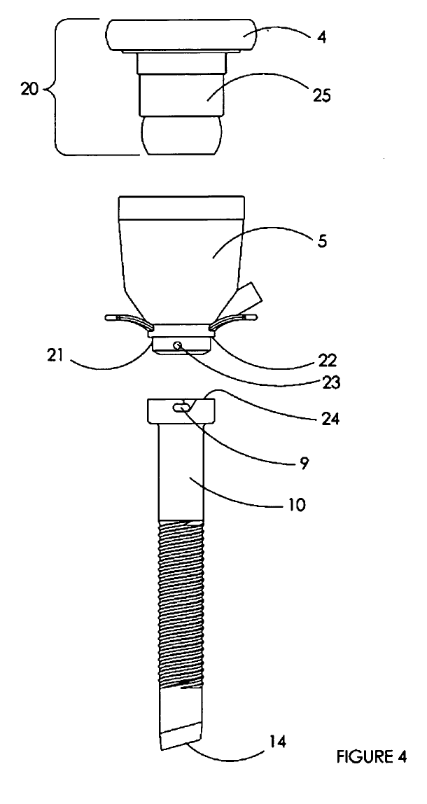

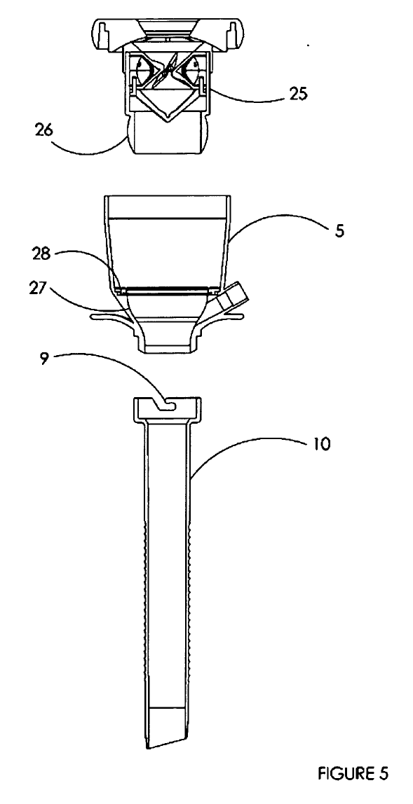

Referring now to FIG. 4, an exploded exterior view of an exemplary cannula according to principles of the invention is shown. Sub-assembly components include a tilting sub-assembly 20, a cap housing 5 and the cannula tube 16. FIG. 5 provides a section view of the subassembly components. When assembled, the tilting lower spherical ball 26 of the tilting sub-assembly 20 is received within the lower spherical ball socket 27 of the cap housing 5 and the outer housing 25 of the tilt assembly is received within the cap housing 5. Advantageously, the ball 26 can pivot and orbit within the socket 27 to provide a range of angular adjustability. Additionally, because the ball 26 is positioned at the end of the tilting sub-assembly 20, a force applied at or near the cap radius flange 4 provides a torque that facilitates angular movement of the tilting sub-assembly 20. A ball positioned near either the proximal end or middle of the tilting sub-assembly 20 would be more difficult to pivot, requiring greater force than the than the tilting subassembly 20 of the invention, and potentially interfering with a procedure. A ball surface gas seal assembly 28, which prevents insufflation gas from escaping to the atmosphere, is pressed and bonded into a seat provided within the cap housing 5.

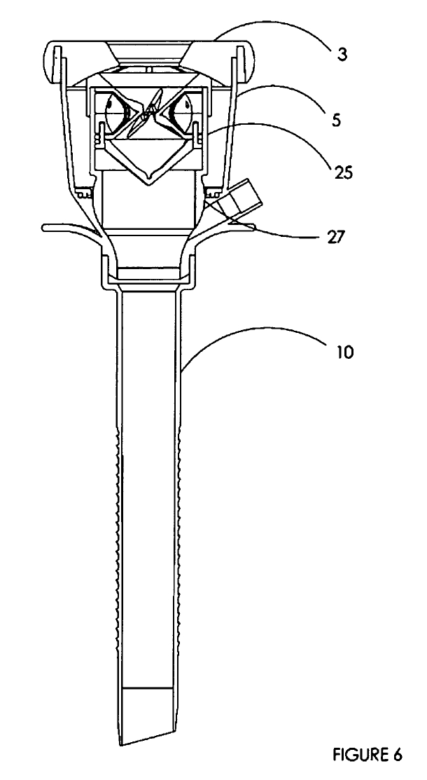

Referring now to FIG. 6, an assembled sectional view of an exemplary cannula according to principles of the invention is shown. The tilting sub-assembly 20 is operably coupled to the cap housing 5 via the ball 26 and socket 27. Additionally, the cap housing 5 is releasably coupled to the cannula tube 16 via twist lock engagement pin 23 and cannula tube twist-lock detail 9.

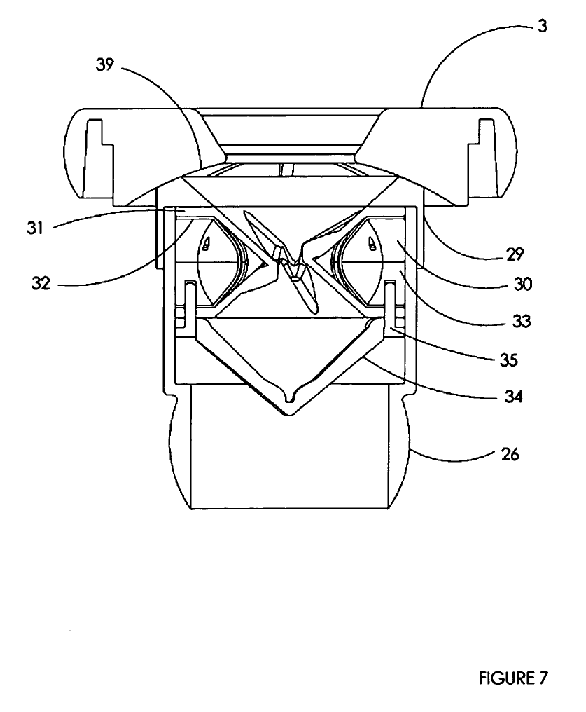

FIG. 7 provides a section view of the tilting sub-assembly 20 with the cap top 3 of the cap housing 5 in a final position on the tilting sub-assembly 20. When installed, the cap top 3 is bonded to the cap housing 5 thereby encapsulating the sub-assembly. An upper spherical ball tilt socket 39 with a center pivot point that is the same as the pivot point used by the lower ball 26 and socket 27, maintains proper working clearance for free orbiting movement of the tilting sub-assembly 20.

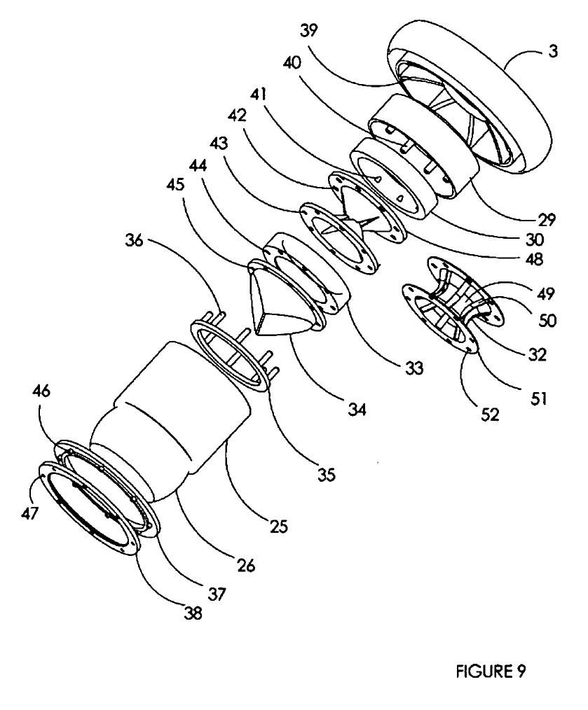

FIGS. 8 and 9 provide exploded side and perspective views of the tilting sub-assembly 20, respectively. The cap top 3 engages an upper spherical ball tilt cap 29, which has a mating upper spherical ball (i.e., convex) surface 29a to facilitate orbital movement of the tilting sub-assembly 20 guided by the cap top 3, which has a corresponding concave surface in contact with the convex surface 29a. Rather than employ a solid surface, the exemplary cap top 3 features an arrangement of ribs to provide a concave surface. However, a solid concave surface would also come within the scope of the invention. During pivotal movement of the tilting sub-assembly 20, the concave surface of the cap top 3 slides on the convex surface 29a of the upper spherical ball tilt cap 29, thereby facilitating free pivotal movement of the tilting sub-assembly 20.

As shown in FIGS. 8 and 9, various components are operatively coupled to form a seal assembly, which includes an instrument seal 31 and a fluid seal. In an exemplary embodiment, flange retainer pins 40 in the spherical ball tilt cap 29 engage opposed flange retainer pins 36 of the fluid seal retainer flange 35. A pair of opposed seal flange retainer rings 30 and 33 are sandwiched in between the spherical ball tilt cap 29 and duckbill retainer flange 35. An instrument seal anti-inversion ring 32 and a concentric resilient instrument seal 31 are sandwiched between the within the pair of opposed seal flange retainer rings 30 and 33. The resilient instrument seal 31 may be folded for mating with the instrument seal anti-inversion ring 32. The instrument seal anti-inversion ring 32 includes a pair of opposed flanges 51 and a plurality of resilient fingers 50 configured to bias the flanges 51 apart. Concomitantly, the flanges 51 of the anti-inversion ring 32 bias the flanges of the hourglass-shaped instrument seal 31 apart. In operation, the instrument seal anti-inversion ring 32 prevents the resilient instrument seal 31 from inverting during withdrawal of an instrument, thereby solving a problem commonly faced by conventional seal assemblies. Inverting can cause leakage of insufflation fluid and result in collapse of an insulated body cavity.

A fluid seal 34 in the form of a duckbill valve is sandwiched between the duckbill retainer flange 35 and a seal flange retainer ring 33. The spherical ball tilt cap 29 engages the tilt assembly outer housing 25. The spherical ball tilt cap 29, the duckbill retainer flange 35 and the components sandwiched therebetween, including the seal flange retainer rings 30 and 33, an instrument seal anti-inversion ring 32 with a concentric instrument seal 31, and a fluid seal 34, comprise a seal assembly, which is enclosed in the tilt assembly outer housing 25 by the spherical ball tilt cap 29. When the device is fully assembled, the lower spherical ball 26 of the tilting sub-assembly 20 is received within the lower spherical ball socket 27 of the cap housing 5. Gas seal 38 and retaining flange 37 comprise a ball surface gas seal assembly 28, which prevents insufflation gas from escaping between the lower spherical ball 26 and the lower spherical ball socket 27 to the atmosphere.

Referring now to FIG. 10, a section view of the seal cap assembly 15 and the tilting sub-assembly 20 is shown in a neutral or centered position. The cap top 3 engages an upper spherical ball tilt cap 29, which has a mating convex surface to guide pivotal movement of the cap top 3. The spherical ball tilt cap 29 engages the tilt assembly outer housing 25. The lower spherical ball 26 of the tilting sub-assembly 20 is received within the lower spherical ball socket 27 of the cap housing 5. Gas seal 38 and retaining flange 37 form a ball surface gas seal assembly, which prevents insufflation gas from escaping between the lower spherical ball 26 and the lower spherical ball socket 27 to the atmosphere. Upwardly angled gas port 6 enables introduction of insulation fluid between the distal end of the device and the fluid seal. Grip wings 7 facilitate manual control.

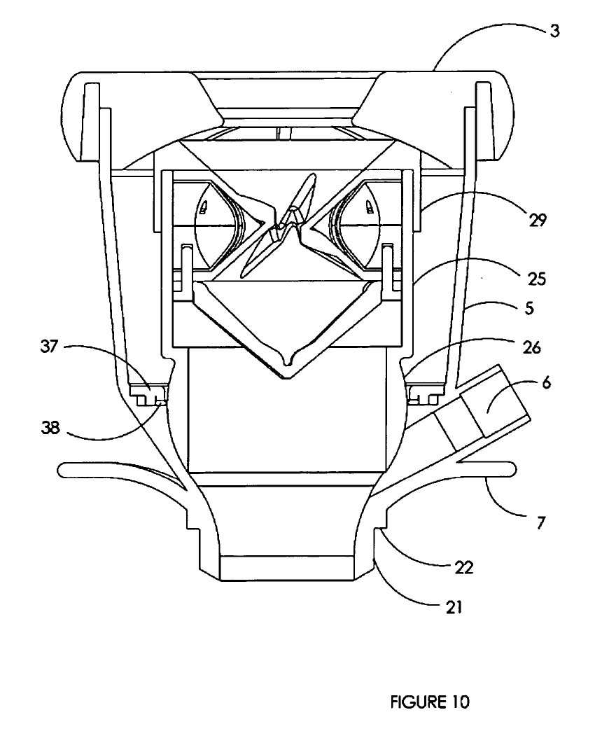

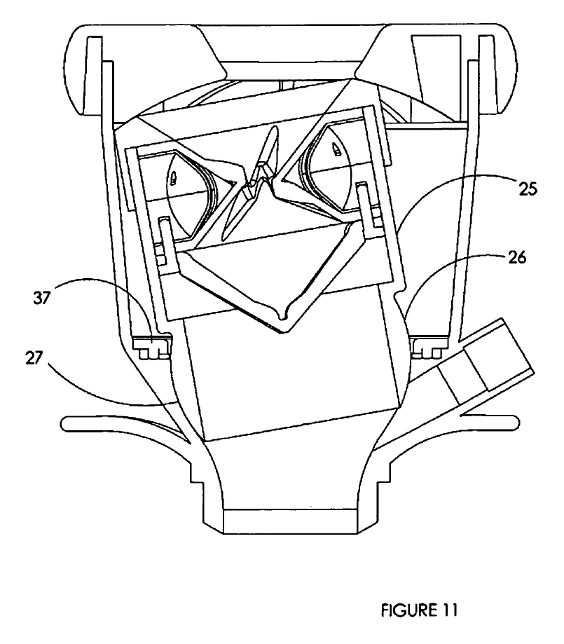

Referring now to FIG. 11, a section view of the seal cap assembly 15 and the tilting sub-assembly 20 is shown in a tilted position. The proximal edge of side wall 31A of the hourglass shaped instrument seal 31 (also referred to herein as an “hourglass instrument seal”) is adjacent to the periphery of the instrument entrance 1. The concave surface of the cap top 3 engages the concave upper spherical surface of the ball tilt cap 29. The spherical ball tilt cap 29 engages the tilt assembly outer housing 25. The lower spherical ball 26 of the tilting sub-assembly 20 is received within the lower spherical ball socket 27 of the cap housing 5. Gas seal 38 and retaining flange 37 form a ball surface gas seal assembly, which prevents insufflation gas from escaping between the lower spherical ball 26 and the lower spherical ball socket 27 to the atmosphere. Those skilled in the art will appreciate that the tilting sub-assembly 20 in the tilted position advantageously allows the hourglass shaped instrument seal 31 to remain in general alignment with the instrument entrance 1, thereby enabling the seal 31 to receive an instrument without inordinate elongation and possible insufflation gas leakage or mechanical failure (e.g., tearing or rupture) of the seal.

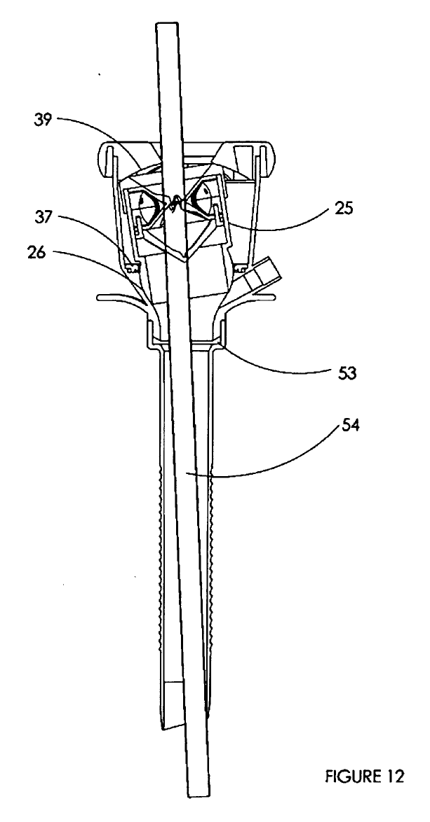

Referring now to FIG. 12, a section view of the complete device with the seal cap assembly 15 and the tilting sub-assembly 20 in a tilted position and a small diameter surgical instrument 54 in place. Those skilled in the art will appreciate that the exemplary seal assembly provides several degrees of pivotal movement of an instrument without causing excessive stress on the exemplary instrument seal. Stress is relieved or minimized because the instrument seal 31 has an hourglass shape which remains in substantial alignment with the entrance 1 and because the tilting sub-assembly 20 pivots.

In an exemplary embodiment, the lower flange 59 of the hourglass-shaped instrument seal 31, the lower seal flange retainer ring 33 and the fluid seal 34 are configured to free-float (i.e., are able to move in a direction parallel to the longitudinal axis of the channel) approximately between the fluid seal retainer flange 35 and the upper flange of the instrument seal anti-inversion ring 32 within the tilt assembly outer housing 25. Such a free floating lower flange of the hourglass instrument seal is referred to herein as a free floating lower flange. The instrument seal anti-inversion ring 32, which includes a pair of opposed flanges 51 and a plurality of resilient fingers 50 configured to bias the flanges 51 apart, bias the flanges of the hourglass-shaped instrument seal 31 apart. The top flange 51 of the anti-inversion ring 32 and the top flange 58 of the instrument seal 31 are fixed in position in the upper seal flange retainer ring 30, while the bottom flange 51 of the anti-inversion ring 32 and the bottom flange 59 of the instrument seal 31 are able to free float. The lower seal flange retainer ring 33 and the fluid seal 34 are also able to free float. Significantly, free floating prevents bunching and binding of the instrument seal 31, which can otherwise compromise the integrity of the seal and interfere with smooth fluid motion of an inserted instrument.

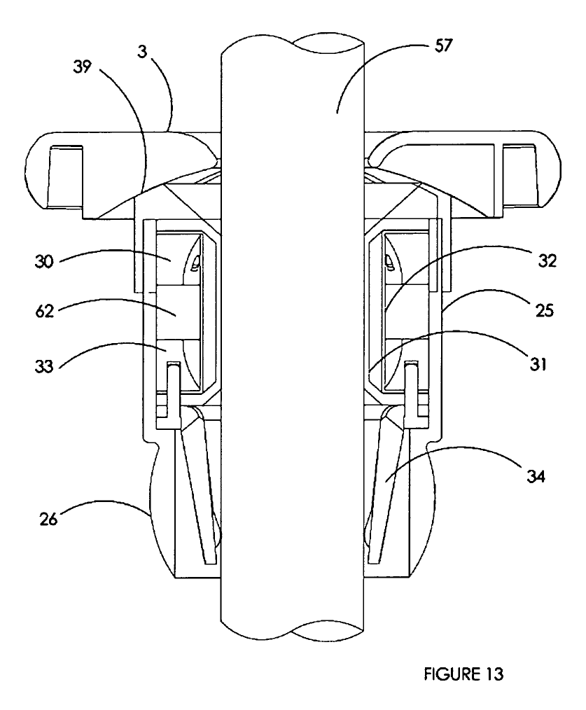

Referring now to FIG. 13, a section view of the tilting sub-assembly 20 with the cap top 3 and a large diameter surgical instrument 57 in place is shown. The inserted instrument 57 causes the fluid (i.e., duckbill) valve 34 to fully open by expanding diametrically. The instrument also displaces the free floating components (i.e., the lower flange 59 of the hourglass-shaped instrument seal 31, the lower seal flange retainer ring 33 and the fluid seal 34) downwardly, thereby extending the resilient fingers 50 of the anti-inversion ring 32 and forming a gap area 62 within the tilt assembly outer housing 25. The floating prevents bunching and binding of the instrument seal 31. Furthermore the instrument seal anti-inversion ring 32 is shown fully dilated. The dilated inversion ring 32 holds the seal extended while the surgical instrument is withdrawn, thereby preventing inversion, bunching and binding of the seal.

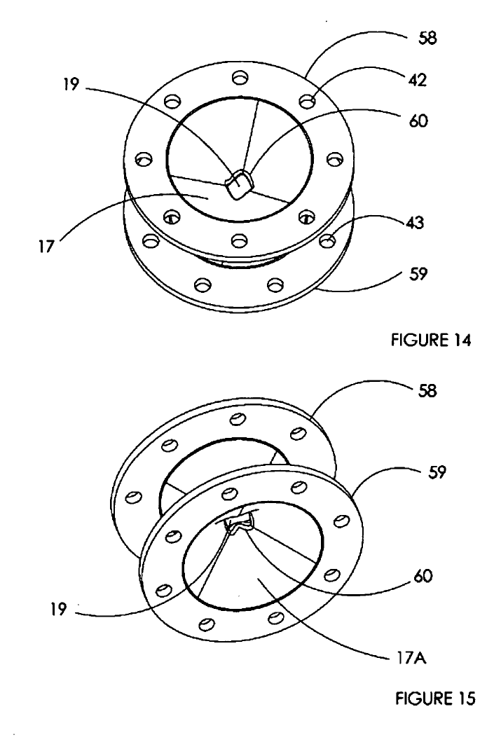

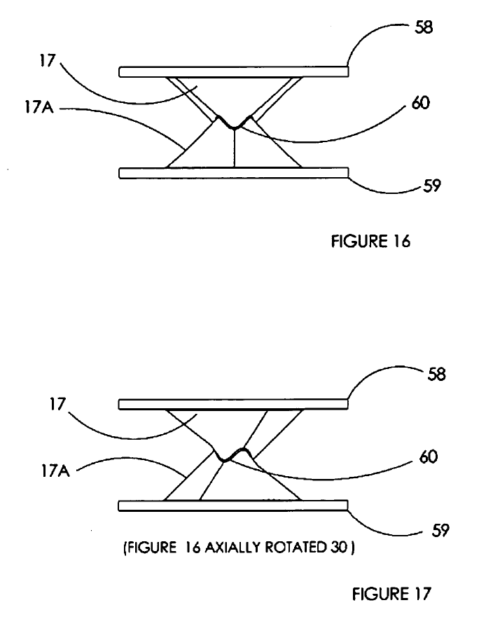

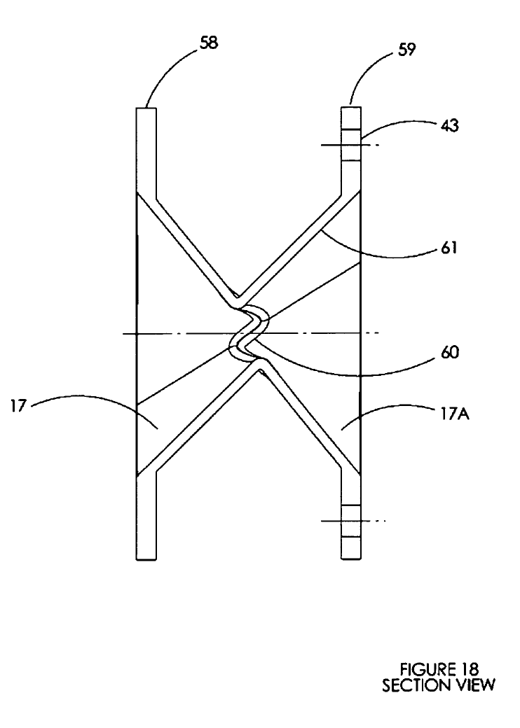

Referring now to FIGS. 14 and 15, perspective views of the top and bottom of the hourglass-shaped instrument seal 31 are provided respectively. The seal includes top (or upper, or proximal end) 58 and bottom (or lower, or distal end) 59 flanges, each having a plurality of apertures (or pin holes) 42 for receiving flange retainer pins 40 and/or opposed flange retainer pins 36. An instrument seal aperture or hole 19 receives an inserted surgical instrument. In general the seal has an hourglass shape, with a top conical surface 17 and a bottom conical surface 17A adjoined at a “trough” or juncture. The trough 60 defines an adjustable aperture which may sealably receive surgical instruments of varying diameters. Advantageously, the rippled shape of the trough 60 allows diametric dilation of the seal without first elongating the elastomer. The trough 60, as more clearly illustrated in the side views of FIGS. 16 and 17 as well as in the section view of FIG. 18, is considered to be rippled. Upon insertion of a surgical instrument having a mid-size diameter, the trough 60 initially flattens out. Upon insertion of a surgical instrument having a large size diameter, the trough 60 flattens and diametrically dilates via elastomer elongation. Use of a rippled trough 60 thus enables the seal 31 to accommodate a wider range of instrument sizes than would otherwise be practicable through dilation alone. Use of a rippled trough 60 also renders unnecessary the armor that is used on many conventional instrument seals to prevent rupture, as is known in the art.

Thus, when a relatively small surgical instrument is inserted, the rippled trough 60 will unfold slightly, causing the seal 31 to stretch slightly, thereby creating an elastic force around the inserted instrument. Consequently, a fluid-tight seal around the surgical instrument is effectuated. Because of the unfolding of rippled trough 60, however, the seal 31 stretches only minimally, thus minimizing the drag force on the surgical instrument and stress and strain on the seal 31. In the case of a surgical instrument with a larger diameter, the rippled trough 60 unfolds to a greater extent than for a smaller surgical instrument and seal 31 stretches. However, because of the accommodation by the unfolded rippled trough, the stress and strain on the seal 31 is minimized. This helps to prevent the drag on the surgical instrument from becoming undesirably high, and the seal from mechanically failing and thereby allowing pressurized insufflation fluid to escape.

With reference again to FIGS. 14 ad 15, the minimum diameter of the aperture 19 should be slightly smaller than the diameter of the shaft of the smallest surgical instrument that the seal 31 is designed to accommodate. By way of example and not limitation, the minimum effective diameter 19 may be about 75% of the diameter of the surgical instrument. The maximum unfolded diameter of aperture 19 is at least equal to the maximum diameter of the largest surgical instrument that the seal 31 is designed to accommodate.

An exemplary hourglass-shape instrument seal 31 is comprised of a flexible material, such as rubber or another elastomeric material. The material should be impervious to air and bodily fluids, should have a high tear strength, and should be flexible. Preferably, the seal is integrally constructed, and is made from a silicone, such as a 50 or 30 durometer shore A liquid silicone rubber. For example, Dow Corning Silastic Q7-4850 liquid silicone rubber may be used. The exemplary hourglass-shape instrument seal 31 may also be made from other silicones, or from materials such as rubber or thermoplastic elastomers. Lubrication may optionally be provided by any suitable lubricant, including fluorosilicone greases and oils. The seal may be impregnated with the lubricant, or, if desired, the seal may also be externally lubricated or lubricated with a surface treatment. Lubrication preferably is provided by coating the surface of the seal with one of the family of parylene compounds such as those available from Specialty Coating Systems, Inc., Indianapolis, Ind. Parylene compounds comprise a family of p-xylylene dimers that polymerize when deposited onto a surface to form a hydrophobic polymeric coating. For example, an instrument seal 31 according to principles of the invention may be coated with polymerized dichloro-(2,2)-paracyclophane (Parylene C) or di-p-xylylene (Parylene N). The Parylene monomers are applied to the surface of the seal by gas-phase deposition in a vacuum chamber.

An exemplary hourglass shaped instrument seal 31 with a rippled trough 60 according to principles of the invention may be made by any number of conventional techniques that are well known to the art. For example, the seal may be molded using liquid injection molding, plastic injection molding, or transfer molding. Preferably, liquid injection molding is used.

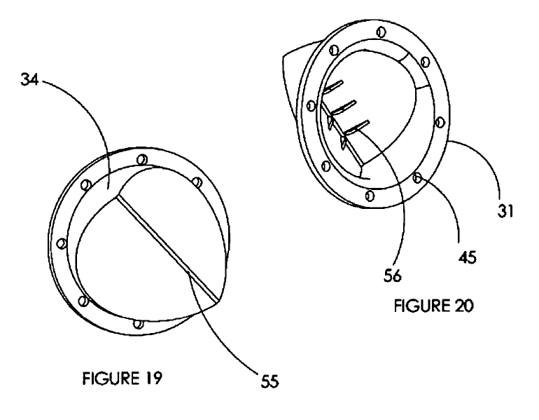

Referring now to FIGS. 19 and 20, bottom and top perspective views of an exemplary fluid seal in the form of a duckbill valve 34 are shown, respectively. The duckbill valve has a pair of resilient flaps separated by a slit 55. The flaps are biased closed when in the relaxed state, but resiliently yield and open when an instrument is pushed through the valve. Advantageously, to guard against inversion, a duckbill valve according to the invention includes a plurality of ribs 56 on each flap, adjacent to and arranged perpendicular to the slit 55. A fluid seal flange 31 and mounting holes 45 are also provided to operable couple the duckbill valve to the cannula.

The conical shape of the upper half of the hourglass-shaped instrument seal 31 assists in guiding a surgical instrument into the cannula. The conical shape provides a funnel effect that directs an instrument to an aperture. While the bottom half of the hourglass-shaped instrument seal 31 does not have to be identical to the top half in size and geometry, such symmetry is preferred to facilitate assembly.

A surgical instrument having seals according to the invention thus overcomes drawbacks of surgical instruments conventional seals. A surgeon may use surgical instruments having a variety of diameters using a single cannula in accordance with principles of the invention. A surgeon may also freely pivot an instrument within the cannula. Further, an hourglass instrument seal according to principles of the invention is inexpensive to manufacture. Moreover, a seal according to the present invention does not require a complex armor mechanisms in order to sealably receive surgical instruments of various diameters.

While the invention has been described in terms of various embodiments, implementations and examples, those skilled in the art will recognize that the invention can be practiced with modification within the spirit and scope of the appended claims including equivalents thereof. The foregoing is considered as illustrative only of the principles of the invention. Variations and modifications may be affected within the scope and spirit of the invention.

What is claimed is:

1. A surgical instrument comprised of a valve seal assembly, said valve seal assembly having an interior and an exterior, and an hourglass instrument seal operably coupled to the interior of the valve seal assembly.

2. A surgical instrument according to claim 1 wherein the hourglass instrument seal includes an upper flange operably coupled to the interior of the valve seal assembly.

3. A surgical instrument according to claim 2 wherein the hourglass instrument seal further includes a free floating lower flange.

4. A surgical instrument according to claim 1 wherein the hourglass instrument seal includes a top conical portion, a bottom conical portion and a rippled junction adjoining the top conical portion and bottom conical portion.

5. A surgical instrument according to claim 1, wherein the valve seal assembly includes a tilt subassembly, a cap housing, a distal end and a proximal end, and
said surgical instrument further comprises a cap top with a concave lower surface disposed at the proximal end of the valve seal assembly, and
the tilt subassembly includes a tilt cap with a convex upper surface adapted to slidably engage the concave lower surface of the cap top, and
the tilt assembly further includes a lower spherical section, and the cap housing includes a ball socket for slidably engaging the lower spherical section of the tilt assembly.

6. A surgical instrument according to claim 1, said valve seal assembly further including a fluid seal, said fluid seal including a duckbill valve, said duckbill valve including a pair of flaps, each flap having a plurality of reinforcing ribs.

7. A surgical instrument according to claim 1, said valve seal assembly having a central channel, a proximal end and a distal end, said surgical instrument including a fluid port operably coupled to said valve seal assembly in fluid communication with said channel, said port having a free end and being disposed at an acute angle relative to the channel, the free end of the port being angled toward the proximal end of the valve seal assembly.

8. A surgical instrument according to claim 1, further comprising a cannula tube operably coupled to the valve seal assembly, said cannula tube including a threaded section including a plurality of independent parallel sets of threads.

9. A surgical instrument according to claim 8, wherein said plurality of independent parallel sets of threads includes triple lead threads.

10. A surgical instrument according to claim 8, wherein said plurality of independent parallel sets of threads start equidistant apart.

11. A surgical instrument according to claim 1 wherein the hourglass instrument seal includes an upper flange operably coupled to the interior of the valve seal assembly, and the hourglass instrument seal is comprised of an elastomer.

12. A surgical instrument according to claim 11 wherein the hourglass instrument seal further includes a free floating lower flange.

13. A surgical instrument according to claim 12 wherein the hourglass instrument seal includes a top conical portion, a bottom conical portion and a rippled junction adjoining the top conical portion and bottom conical portion.

14. A surgical instrument according to claim 13, wherein the valve seal assembly includes a tilt subassembly, a cap housing, a distal end and a proximal end, and
said surgical instrument further comprises a cap top with a concave lower surface disposed at the proximal end of the valve seal assembly, and
the tilt subassembly includes a tilt cap with a convex upper surface adapted to slidably engage the concave lower surface of the cap top, and
the tilt assembly further includes a lower spherical section, and the cap housing includes a ball socket for slidably engaging the lower spherical section of the tilt assembly.

15. A surgical instrument according to claim 14, said valve seal assembly further including a fluid seal, said fluid seal including a duckbill valve, said duckbill valve including a pair of flaps, each flap having a plurality of reinforcing ribs.

16. A surgical instrument according to claim 15, said valve seal assembly having a central channel, a proximal end and a distal end, said surgical instrument including a fluid port operably coupled to said valve seal in fluid communication with said channel, said port having a free end and being disposed at an acute angle relative to the channel, the free end of the port being angled toward the proximal end of the valve seal assembly.

17. A surgical instrument according to claim 16, further comprising a cannula tube operably coupled to the valve seal assembly, said cannula tube including a threaded section including a plurality of independent parallel sets of threads.

18. A surgical instrument according to claim 17, wherein said plurality of independent parallel sets of threads includes triple lead threads.

19. A surgical instrument according to claim 18, wherein said plurality of independent parallel sets of threads start equidistant apart.

20. A surgical instrument according to claim 3, further comprising an anti-inversion member configured to bias the top flange apart from the bottom flange of the hourglass instrument seal.
